(19) United States
(12) Patent Application Publication
(10) Pub. No.: US 2012/0252118 A1
(43) Pub. Date: Oct. 4, 2012

(54) PLASTIC PRESSURE VESSEL FOR BIOPHARMACEUTICAL APPLICATIONS AND METHODS THEREOF

(76) Inventors: Jean-Pierre R. Karam, Wyckoff, NJ (US); James J. Nason, Springfield, MA (US); Richard D. Sale, Tolland, CT (US)

(21) Appl. No.: 13/515,019

(22) PCT Filed: Aug. 9, 2010
(86) PCT No.: PCT/US2010/044842

§ 371 Date: Jun. 11, 2012

Related U.S. Application Data
(60) Provisional application No. 61/287,278, filed on Dec. 17, 2009.

(51) Int. Cl. B65D 85/00; C12N 1/20; B29C 39/38; C07K 1/34; C07H 1/06; B29C 39/02; C12N 5/07; C12N 1/16

(52) U.S. Cl. 435/348; 435/325; 206/524.1; 435/252.1; 435/255.1; 530/412; 536/25.4; 264/299

(57) ABSTRACT

Described herein is a molded plastic pressure vessel for biopharmaceutical applications and methods thereof. The molded plastic pressure vessel has a surface area of at least 500 inches2 and comprises a polyphenylene oxide polymer; and at least one antioxidant.

### TECHNICAL FIELD

A molded plastic pressure vessel for biopharmaceutical applications with a large surface area is described. Methods of making and using the molded plastic pressure vessel are also described.

### BACKGROUND

Bioprocessing filtration has traditionally been done with vertically stacked filter elements placed in stainless steel vessels. Typically, the stainless steel vessel serves to contain the system pressure. The filter elements can be difficult to assemble and disassemble into and out of the apparatus as they are usually placed over a long center mandrel connected to the apparatus base. This may require use of overhead assisted lifting devices, particularly after the product is used, since when it is wet the cartridges tend to be heavy. This process is also typically messy as the residual process liquid is not contained, which can lead to exposure of bio-hazardous liquid(s) to the user. In addition, the apparatus needs to be fully cleaned after use to eliminate cross-contamination exposure. This requires time, effort, cleaning agents (which are not environmentally friendly), and the potential of cross-contamination between lots of material if the equipment is not cleaned adequately. For at least these reasons, the industry has been shifting toward the use of fully disposable products (e.g., polymer-based products) that allow any component that touches the process fluid to be disposed of For example, disposable filtration systems are described in U.S. Prov. Pat. Appl. 61/256643 (Bryan et al.) and U.S. Pat. Appl. Publ. No. 2005/0279695 (Straeffer et al.).

### SUMMARY

There is a need to identify a molded plastic pressure vessel that may be used in biopharmaceutical applications, which is chemically resistant, structurally adequate, weldable, autoclavable, has an acceptable extractable and leachable profile, and optionally, is aesthetically acceptable.

In one aspect, the present disclosure provides a molded plastic pressure vessel for biopharmaceutical applications comprising: a polyphenylene oxide and at least one antioxidant, wherein the molded plastic pressure vessel has a surface area of at least 500 inches².

In some embodiments, the molded plastic pressure vessel for biopharmaceutical applications further comprises a white colorant.

In some embodiments, the molded plastic pressure vessel for biopharmaceutical applications further comprises polystyrene.

In some embodiments, a method of filtering a biopharmaceutical matrix is disclosed comprising providing a molded pressure vessel as disclosed herein, wherein the molded pressure vessel contains a filter medium; and filtering a biopharmaceutical fluid through the filter medium.

In another aspect, a method of making a molded plastic pressure vessel for biopharmaceutical applications is disclosed comprising: contacting a mold comprising a cavity having a surface area of at least 500 inches²with molten mixture comprising polyphenylene oxide and at least one antioxidant; and cooling the molten mixture to form the molded plastic pressure vessel.

In some embodiments, the molded plastic pressure vessel does not exhibit yellowing as compared to a molded article made by an identical process wherein the molten mixture is free of any antioxidant.

The above summary is not intended to describe each embodiment. The details of one or more embodiments of the invention are also set forth in the description below. Other features, objects, and advantages will be apparent from the description and from the claims.

### BRIEF DESCRIPTION OF THE DRAWINGS

FIG. 1 is a schematic of vessel used in biopharmaceutical applications.

### DETAILED DESCRIPTION

As used herein, the terms

“a”, “an”, and “the” are used interchangeably and mean one or more; and

“and/or” is used to indicate one or both stated cases may occur, for example A and/or B includes, (A and B) and (A or B).

Also herein, recitation of ranges by endpoints includes all numbers subsumed within that range (e.g., 1 to 10 includes 1.4, 1.9, 2.33, 5.75, 9.98, etc.).

Also herein, recitation of “at least one” includes all numbers of one and greater (e.g., at least 2, at least 4, at least 6, at least 8, at least 10, at least 25, at least 50, at least 100, etc.).

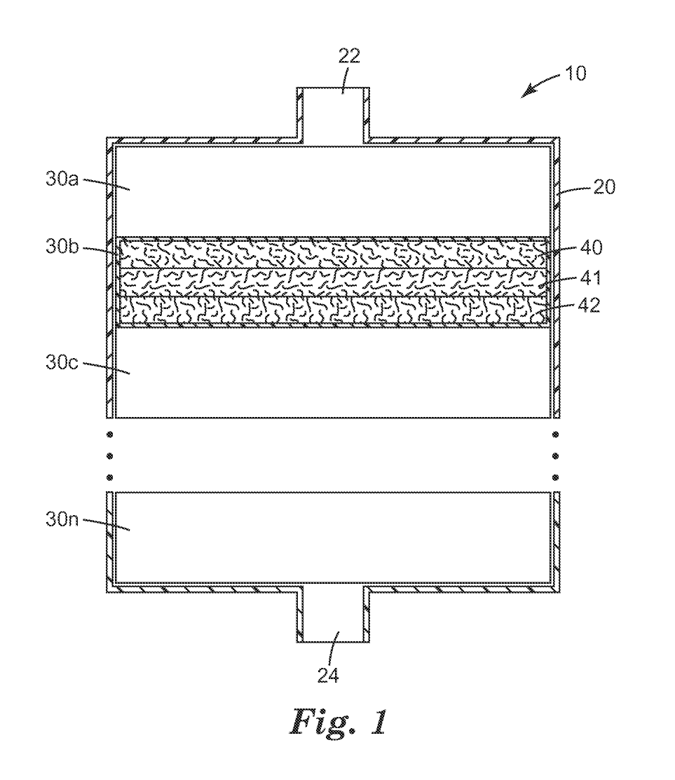

In filtration technology it is known to stack shells, which support and encapsulate filter media, to create apparatuses to filter and/or purify fluids. These shells and apparatuses differ in design and operation. To define the terminology used herein, shown in FIG. 1 is apparatus 10, which is used to filter fluids. Apparatus 10 comprises inlet 22 and outlet 24, which are used for fluid entry and exit, respectively.

The apparatus comprises a molded plastic pressure vessel. In one embodiment, the multiple molded plastic pressure vessels are stacked as shown in FIG. 1. Apparatus 10 comprises molded plastic pressure vessels 30a, 30b, 30c . . . 30n contained within optional housing 20. The molded plastic pressure vessel comprises one filter element, which is used to filter fluid. In one embodiment, the molded plastic pressure vessel comprises more than one filter element. For example, molded plastic pressure vessel 30b in FIG. 1 comprises three filter elements, 40, 41, and 42. In one embodiment, the filter elements are the same. In another embodiment, the filter elements are different. FIG. 1 is for illustrative purposes only and should not be used to unduly limit the present disclosure. For example, although not shown, the filter element may be a lenticular filter and/or the inlet and outlet may be co-located on the apparatus.

The filter elements (e.g., filter medium) not only have a screening effect whereby coarse particles are retained on the surface of the filter elements, but also, in particular, a depth effect for fine particles, which are retained in the void spaces within the filter material. Depending on the type of materials used, these filter elements may, for example, also have an adsorbing effect or interact with the unfiltered fluid in another way which goes beyond the purely mechanical filtration effect. Moreover, the surface may be subsequently treated for certain applications so as to prevent detachment of fibrous particles in the dry and damp states. Filter active materials are known to those skilled in the art and include, for example, perlites, kieselguhrs, fibrous materials, and adsorbents such as activated carbon polyvinyl pyrrolidine and polyvinyl pyrrolidine-iodine substances. The filter elements, which are porous structures having porosities between about 50-80% by volume, may be relatively fragile.

The present disclosure is directed to the molded plastic pressure vessels, which are used for mechanical support and reinforcement of the filter media. Examples of molded plastic pressure vessels include those commercially available in the following filtration apparatuses: “ZETA PLUS ENCAPSULATED SYSTEM” by 3M Purification Inc., St. Paul, Minn.; “MILLISTAK+POD DISPOSABLE DEPTH FILTER SYSTEM” by Millipore Corp., Billerica, Mass.; and “STAX DISPOSABLE DEPTH FILTER SYSTEM” by Pall Corp., Port Washington, N.Y.

The molded plastic pressure vessel disclosed herein is resistant to corrosive materials, structurally adequate, weldable, autoclavable, has an acceptable extractable and leachable profile, and optionally, is aesthetically acceptable.

The molded plastic pressure vessel of the present disclosure comprises a polymeric composition comprising polyphenylene oxide (PPO, also called polyphenylene ether). Polyphenylene oxide is a linear amorphous polymer. Polyphenylene oxide may be formed via oxidative coupling of a phenolic compound (e.g., 2,6-dimethylphenol). This polymer may be blended with other polymers such as, polyamides, polystyrene, high impact polystyrene, or polyolefins to create thermoplastic resins. Exemplary polyolefins include: polybutadiene, polyethylene, polypropylene, and combinations thereof. Generally, the polymer blend comprises at least 1, 2, 4, 5, 10, 20, 25, 30, 40, or even 50 parts by weight of polyphenylene oxide; at most 50, 60, 70, 75, 80, 90, 95, or even 99 parts by weight of polyphenylene oxide per 100 parts total weight of polymer present.

Commercially available blended polyphenylene oxide is available, for example, under the trade designations “NORYL GFN”, or “NORYL PPX” from SABIC Innovation Plastics Holdings, Brampton, Ontario; “XYRON” from Asahi Kasei, Fowlerville, Mich.; and “LURANYL” from BASF Corp., Florham Park, N.J.

Particularly useful, is a polymeric composition comprising a blend of a polyphenylene oxide polymer and polystyrene, which forms a thermoplastic resin referred to as a modified polyphenylene oxide. These modified polyphenylene oxides have been shown to be weldable and autoclavable. Further, the modified polyphenylene oxide was found to withstand exposure to chemicals such as caustic solutions, which may be used in biopharmaceutical applications for pre- or post-filtration sanitization.

To provide structural support to the pressure vessel, reinforcing fillers may be added to the polymeric composition. Reinforcing fillers, in amounts sufficient to impart reinforcement, can be used, for example, metals such as aluminum, iron or nickel; and non-metals, such as, for example, carbon filaments, silicates, potassium titanate, titanate whiskers, and glass particles. Exemplary glass particles include: flakes, fibers (or whiskers), bubbles, and beads. The glass may comprise lime-aluminum borosilicate glass that is relatively soda free (known as “E” glass) or low soda glass (known as “C” glass). The reinforcing fillers may be added in amounts of at least 5, 10, 15, 20, 25, or even 30% by weight; at most 20, 25, 30, 40, 50, or even 60% by weight based on the combined weight of the reinforcing filler and the polymer.

The polymeric composition may comprise other monomers or fillers to improve processibility or the mechanical or chemical properties of the finished product. Fillers include those known in the art; for example, talc, mica, clay, magnesium hydroxide, potassium titanate, carbon (fibers or particles), calcium carbonate, etc. Fillers may be added in an amount of at least 1, 2, 5, 10, 20, 30, 40 or even 50% by weight; at most 10, 15, 20, 25, 30, 40, 60, 80, or even 100% by weight based on the total weight of polymer present.

In some embodiments, the composition does not contain a flame retardant or contains less than 0.01 percent of a flame retardant, while in others, the composition comprises flame retardants, such as for example non-halogenated and halogenated compounds. Exemplary halogenated compounds include: chlorinated and brominated phosphates (e.g., brominated phosphate ester, polybrominated aryl phosphates, etc.).

Typically, commercial modified polyphenylene oxide resins are available in natural (yellow-brown), black, blue, and grey colors. Because the molded articles disclosed herein are used in biopharmaceutical applications (e.g., clean rooms, good manufacturing practice (GMP) facilities, etc.) having aesthetically pleasing components is desired. Thus, an opaque white colorant may be added to the polymeric composition. Exemplary white colorants include titanium dioxide, zinc oxide, zinc sulfide, barium sulfate, calcium carbonate, and talc. These white colorants are added to the polymeric composition in quantities no greater than 2, 4, 5, 6, 8, 10, or even 20% by weight based on the total weight of polymer present.

It was found upon molding of the modified polyphenylene oxide with white colorant that the resulting article appeared to have a yellow discoloration. Thus, an antioxidant was added to the polymeric composition prior to molding to inhibit yellowing. Exemplary antioxidants include hindered phenols, phosphites, thioesters, and amines.

Exemplary hindered phenols include 2,6,di-tert-butyl-4-methylphenol, commonly known as BHT (sold under the trade designations Ionol by Shell Chemical Co.); 4,4-methylene bis(2,6-di-tert-butylphenol) and 2,6-di-tert-butyl-4-n-butylphenol; tetrakis[methylene 3-(3′,5′-di-tert-butyl-4′-hydroxyphenyl) propionate]methane; stearyl-3-3-(3′,5′-di-tert-butyl-4′-hydroxyphenyl)propionate; N, N′-hexane-1,6-diylbis[3-(3,5-di-tert-butyl)-4-hydroxyphenyl]propionamide; N,N′-bis(3,5-di-butyl-4-hydroxyl-phenylpropionyl) hydrazine; and tris(3,5-di-tert-butyl-4-hydroxybenzyl)isocyanurate (sold under the trade designations “IRGANOX 1010”, “IRGANOX 1076’, “IRGANOX 1098”, “IGANOX MD-1024”, and “IGANOX 3114”, respectively by Ciba-Geigy Corp., Dover Township, NJ); 2,4,6-tris-(3′-5-di-tert-butyl-4-hydroxybenzyl)-1,3,5-triazine; 2,2-methylenebis(4-methyl-6-tert-butylphenol; 4,4′-butylidenebis(3-methyl-6-tert-butylphenol), n-octadecyl 3-(3,5-di-tert-butyl-4-hydroxyphenyl)propionate; 1,1,3-tris(2-methyl-4-hydroxy-5-tert-butylphenyl)butane; pentaerythritol; tetrakis[3-(3,5-di-tert-butyl-4-hydroxyphenyl)propionate]; tocopherols (e.g., Vitamin E), and combinations thereof.

Exemplary phosphites include: tri-(2, 4-ditertbutyl phenyl) phosphate and bis (2,4-ditertbutyl phenyl) pentaerythritol diphosphite (sold under the trade designations “ALKANOX 240” and “ULTRANOX 626” from Chemtura Corp., Middlebury, Conn.); tris(nonylphenyl phosphate and distearylpentaerythitol diphosphite); and combinations thereof.

Exemplary thioesters include: dilauryl thiodipropionate and distearyl thiodipropionate (sold under the trade designations “IRGANOX PS800” and “IRGANOX PS802”, respectively by Ciba-Geigy Corp.); dimyristyl thiodipropionate; thiodipropionic acid, and combinations thereof.

Exemplary amines include: butylated octylated diphenylamine; nonylated diphenylamine; (sold under the trade designations “IRGANOX L-57” and “IRGANOX L-67”, respectively by Ciba-Geigy Corp.); 4,4′-dioctyl diphenylamine; (sold under the trade designations “VANOX-12” by ThermoFisher Scientific, Waltham, Mass.); dicumylated diphenylamine; (sold under the trade designations “PERMANAX CD”); and combinations thereof.

In one embodiment, the antioxidants may be blended, for example the hindered phenols can also be admixed with phosphates (e.g., catechol-2,6-di-tert-butyl-4-methyl-phenol-phosphite), and/or alkylamines (e.g., dimethyloctadecylamine).

The antioxidant is added to the polymeric composition comprising the polyphenylene oxide in a sufficient quantity to prevent yellowing as determined by CIE colorspace and the calculated yellowness index (YI) following ASTM E313-98. In one embodiment, the amount of antioxidant added is at least 0.5, 1, 2, 4, 5, 6, or even 8%; at most 2, 4, 5, 6, 8, 10, or even 12% by weight based on the total weight of polymer. In one embodiment, the molded plastic pressure vessel of the present disclosure does not exhibit yellowing as compared to a molded article made by an identical process wherein the molten mixture is free of any antioxidant. For example, the YI between two molded articles made by an identical process, wherein the molten mixture comprises at least one antioxidant and the other molder mixture is free of antioxidant, differ by more than 0.3, 0.5, 1.0, 1.5, 2, 2.5, or even 3.

The polymeric compositions are molded using molding processes as are known in the art, for example injection molding processes. Other exemplary molding methodologies include, thermoforming, transfer molding, rotational molding, reaction injection molding, compression molding, extrusion, liquid casting, selective laser sintering, and stereolithography.

In another embodiment, in addition to or in place of the antioxidant, an ascetically-pleasing molded plastic pressure vessel may be formed by purging the mold with an inert gas prior to molding of the polyphenylene oxide.

An inert gas, such as argon, helium, or nitrogen may be used. The inert gas is introduced into the mold using techniques known to those of ordinary skill in the art to evacuate the mold chamber of air and oxygen.

For example, the inert gas may be supplied through vents in the mold chamber to evacuate the air as the mold is preheated. The inert gas may be pushed into the mold via pressure or pulled into the mold via a vacuum. The inert gas flows through the mold for a predetermined time to evacuate substantially all air and oxygen from the mold chamber. After evacuation, the molten polyphenylene oxide resin is introduced into the evacuated mold chamber and then subsequently hardened.

As is known to those of ordinary skill in the art, the mold chamber may comprise vents, which allow the evacuated air and/or oxygen to vent and later, when filling the mold chamber, allow the inert gas to vent as the molten resin fills the mold chamber. If pressure is used, the inert gas may be introduced at pressures between 1 and 2000 psi (pounds per square inch) with lower pressures between 1 and 20 psi being preferred because less gas is used. If a vacuum is used, a vacuum in the range of 15 inHg (inches mercury) to about 29 inHg may be sufficient to evacuate the air and oxygen from mold chamber.

These molded plastic pressure vessels may be used in biopharmaceutical applications to remove impurities or to isolate a particular analyte or analytes. Exemplary applications include: clarification of mammalian cell culture harvests, clarification of bacteria, yeast and incest cell lysates, host cell protein removal, virus and DNA reduction, protein aggregate removal, and endotoxin removal.

As mentioned above, the molded plastic pressure vessel is used to provide mechanical support and reinforcement to a filter medium. Generally, the molded plastic pressure vessel comprises a plurality of ribs extending radially outward from a central hub in a spoke-like fashion, such as those disclosed in U.S. Pat. No. 4,783,262 (Ostreicher et al.), however, any rib geometry may be used to effectively support the filter medium and not impede to flow of fluid through the vessel. The molded polymer of the molded plastic pressure vessel is substantially nonporous (i.e., fluid generally does not leak through the modified polyphenylene oxide of the molded plastic pressure vessel).

In one embodiment, the molded plastic pressure vessels are used to filter large quantities of biopharmaceutical fluids. Because these fluids may contain a high volume of solids, typically the filter medium has a large surface area. The molded plastic pressure vessels of the present disclosure have a surface area of at least 2581, 3226, 3871, 4839, or even 6452 cm² (400, 500, 600, 750, or even 1000 inches²); at most 6452, 12903, 25807, 38710, or even 51613 cm² (1000, 2000, 4000, 6000, or even 8000 inches²).

Molded plastic pressure vessels as disclosed herein may encapsulate filter media that are made of flat materials, for example, filter cardboards, papers, tiles, or fabrics. The molded plastic pressure vessels may be used in depth filtration systems. A depth filtration system comprises a depth filter (comprising organic and/or inorganic fibrous and/or granular substances), which is a filter medium that retains particles throughout the medium as opposed to just the surface of the medium. Depth filtration systems are commonly used in biopharmaceutical applications, because of their ability to filter liquids comprising a high particle load without becoming clogged.

In one embodiment of the present disclosure, a depth filter is placed inside of a molded pressure vessel as disclosed herein to form a depth filtration system. A biopharmaceutical fluid (such as those disclosed above) is then passed through the depth filtration system using techniques known in the art (e.g., back pressure, gravity, centrifugal force, etc.) so as to filter (or purify) the biopharmaceutical fluid.

### EXAMPLES

Advantages and embodiments of this disclosure are further illustrated by the following examples, but the particular materials and amounts thereof recited in these examples, as well as other conditions and details, should not be construed to unduly limit this invention. In these examples, all percentages, proportions and ratios are by weight unless otherwise indicated.

Table of Materials

| Name | Description |
|---|---|
| PPO | Modified polyphenylene oxide resin comprising a blend |
| | of polyphenylene ether and polystyrene, 30% by weight |
| | glass fiber and a white color concentrate, available under |
| | the trade designation “NORYL GFN3 RESIN” from |
| | SABIC Innovative Plastics, Pittsfield, MA. The PPO |
| | blend was dried in a desiccant air dryer (Model # |
| | D01A4000000 equipped with a Compu-Dry CD 100 |
| | Controller; commercially available from Conair Group |
| | Inc, Cranberry Township, PA) at 200° F. (93.3° C.) for 4 |
| | to 6 hours. |
| Antioxidant 1 | Propanoic acid, 3-(dodecylthio)-,2,2-bis[[3-dodecylthio)- |
| | 1-oxopropoxy]methyl]-1,3-propanediyl ester available |
| | under the trade designation “SEENOX 412S” from |
| | Shipro Kasei Kaisha LTD., Osaka, Japan. |
| Antioxidant 2 | Tris(2,4-di-t-butylphenul) phosphite available under the |
| | trade designation “ALKANOX 240” from Chemtura |
| | Corp., Middlebury, CT. |

### Injection Mold 1

An injection mold was made of 420 stainless steel using different machining methods such CNC machining, EDM, grinding and was manually polished. The mold was used to produce a pressure vessel similar to that disclosed in, for example FIGS. 9 and 10, of U.S. Prov. Appl. No. 61/256,643 (Bryan et al.). The cavity of the mold had an approximately surface area of 6,450 cm² (1000 in²) and an approximate volume of 1,606 cm³ (98 in³).

### Injection Mold 2

An injection mold was made of 420 stainless steel using different machining methods such CNC machining, EDM, grinding and was manually polished. The mold was used to produce a 8.3 cm (3.28 in) diameter O-ring retainer, having a height of 11.1 mm (0.440 in) and a maximum wall thickness of 4.8 mm (0.190 in). The cavity of the mold had an approximately surface area of 90 cm² (13.9 in²) and an approximate volume of 8 cm³ (0.5 in³) volume.

### Colorimetry Method

Two, and in the case of Comp. Ex. A which was three, relatively flat samples were cut from the molded plastic pressure vessel and the molded O-ring retainer was used as is. A colorimeter sold under the trade designation “X-RITE SP64” sphere spectrophotometer/color meter (X-Rite USA, Grand Rapids, Mich.) was used. The spectrometer was recalibrated (zeroed to white reference and black reference) immediately before use. The Specular Excluded (SPEX) Reflectance data was collected and YI was reported following ASTM E313-98 using a CIE Source D65 with a 10 degree observer and a 4 mm spot size, also reported was the L*a*b* colorspace values. Shown in Table 2 are the results.

### Examples 1-5 and Comparative Example A (Comp. Ex. A)

PPO and antioxidant(s), if used, were dry blended as described in Table 1, where kg is kilograms, g is gram, and lb is pound. The dry blended material was placed into a 610 Tons Toyo injection molding machine (commercially available from Plastixs LLC, Shrewsbury, Mass.) and heated to approximately 299° C. (570° F.). The molten resin was injected into Injection Mold 1, which was previously heated to 82° C. (180° F.). After filling the mold with the molten polymer, water circulating through the mold was used to cool down the mold and molded article. After cooling, the mold was opened and the molded article was manually extracted from the mold and put on a flat table to cool to room temperature. The resulting molded articles were visually observed and tested following the Colorimetry Method as described above. The results are reported in Table 1 below.

### Comparative Example B (Comp. Ex. B)

PPO was placed into an injection molding machine (610 Tons Demag injection molding machine manufactured by Van Dorn) and heated to 299° C. (570° F.). The molten resin was injected into Injection Mold 2 at a rate of 3 inches per second (7.6 centimeters per second). After filling the mold with molten polymer, the mold was cooled to 93° C. (200° F.) with water. After cooling, the mold was opened and the molded article was ejected into a plastic lined bin and cooled to room temperature. The resulting molded article was visually observed and tested following the Colorimetry Method as described above. The results are reported in Table 1, below.

TABLE 1

| | PPO | Antioxidant | Antioxidant | |
|---|---|---|---|---|
| Example | kg (lb) | 1 g (lb) | 2 g (lb) | Visual |
| Comp. | 45 (100) | 0 | 0 | Yellowish brownish |
| Ex. A | | | | in color, splotchy |
| | | | | yellow appearance |
| 1 | 45 (100) | 227 (0.50) | 0 | Very slight |
| | | | | improvement versus |
| | | | | Comp. Ex. A |
| 2 | 45 (100) | 0 | 227 (0.50) | Some color |
| | | | | improvement versus |
| | | | | Comp. Ex. A |
| 3 | 45 (100) | 113 (0.25) | 113 (0.25) | Very slight color |
| | | | | improvement versus |
| | | | | Comp. Ex. A |
| 4 | 45 (100) | 0 | 454 (1) | A big color |
| | | | | improvement, |
| | | | | appears much whiter |
| | | | | than Comp. Ex. A. |
| 5 | 45 (100) | 227 (0.50) | 227 (0.50) | Some color |
| | | | | improvement versus |
| | | | | Comp. Ex. A |
| Comp. | 45 (100) | 0 | 0 | Appears white with, |
| Ex. B | | | | no yellow spots. |

TABLE 2

| Example | L* | A* | B* | YI |
|---|---|---|---|---|
| Comp. Ex. A | 84.0 | −2.43 | 4.78 | 7.9 |
| | 84.4 | −2.40 | 4.04 | 6.4 |
| Comp. Ex. A | 84.1 | −2.50 | 6.05 | 10.4 |
| | 84.8 | −2.45 | 4.38 | 7.0 |
| Comp. Ex. A | 83.9 | −2.37 | 5.80 | 10.0 |
| | 84.8 | −2.45 | 4.39 | 7.0 |
| 1 | 84.4 | −1.88 | 4.63 | 8.0 |
| | 84.4 | −1.70 | 4.31 | 7.5 |
| 1 | 84.7 | −1.87 | 4.62 | 8.0 |
| | 85.1 | −1.75 | 4.20 | 7.2 |
| 2 | 84.2 | −1.32 | 3.93 | 7.1 |
| | 84.6 | −1.16 | 3.31 | 5.9 |
| 2 | 82.9 | −1.02 | 4.73 | 9.1 |
| | 84.6 | −1.19 | 3.45 | 6.2 |
| 3 | 85.3 | −2.21 | 3.54 | 5.5 |
| | 85.1 | −1.99 | 3.36 | 5.3 |
| 3 | 84.9 | −1.95 | 3.72 | 6.1 |
| | 85.4 | −2.14 | 3.24 | 4.9 |
| 4 | 85.0 | −1.70 | 2.31 | 3.4 |
| | 85.0 | −1.65 | 2.35 | 3.5 |
| 4 | 85.1 | −1.75 | 2.51 | 3.8 |
| | 85.1 | −1.68 | 2.19 | 3.2 |
| 5 | 85.0 | −1.99 | 3.25 | 5.1 |
| | 84.8 | −2.08 | 3.37 | 5.3 |
| 5 | 85.0 | −2.06 | 3.54 | 5.6 |
| | 85.1 | −2.13 | 3.65 | 5.8 |
| Comp. Ex. B | 79.8 | −1.15 | 4.00 | 7.7 |
| | 80.6 | −1.18 | 3.89 | 7.4 |
| Comp. Ex. B | 82.4 | −1.11 | 4.05 | 7.7 |
| | 82.5 | −1.14 | 3.98 | 7.5 |

As shown in Tables 1 and 2, above, Comparative Example B appeared white, however upon quantitative testing was found to exhibit some yellow color. Comparative Example A, appeared visually yellow with apparent yellow spots throughout the surface of the molded article. As shown in Table 1, the addition of antioxidant to the polymeric composition in Examples 1-5 assisted in reducing the YI value and, in some instances, made the color of the resulting article more uniform.

Foreseeable modifications and alterations of this invention will be apparent to those skilled in the art without departing from the scope and spirit of this invention. This invention should not be restricted to the embodiments that are set forth in this application for illustrative purposes.

1. A molded plastic pressure vessel for biopharmaceutical applications comprising:
a polyphenylene oxide; and
at least one antioxidant;
wherein the molded plastic pressure vessel has a surface area of at least 500 inches².

2. The molded plastic pressure vessel of claim 1, further comprising polystyrene.

3. The molded plastic pressure vessel of claim 1, further comprising a white colorant.

4. The molded plastic pressure vessel of claim 1, further comprising glass particles.

5. The molded plastic pressure vessel of claim 1, wherein the at least one antioxidant is added in a sufficient quantity to prevent yellowing as determined by the yellowness index according to ASTM E313-98.

6. The molded plastic pressure vessel of claim 1, wherein the at least one antioxidant is selected from one of: tetrakis[methylene-3-(3′,5′-di-tert-butyl-4′-hydroxyphenyl) propionate]methane, distearyl thiodipropionate, dilauryl thiodipropionate, thiodipropionic acid, and combinations thereof.

7. A method of filtering a biopharmaceutical matrix comprising:
providing a molded pressure vessel according to claim 1, wherein the molded pressure vessel contains a filter element; and
filtering a biopharmaceutical fluid through the filter element.

8. A method of making a molded plastic pressure vessel for biopharmaceutical applications comprising:
contacting a mold comprising a cavity having a surface area of at least 500 inches² with a molten mixture comprising polyphenylene oxide and at least one antioxidant; and
cooling the molten mixture to form the molded plastic pressure vessel.

9. The method of claim 8, wherein the molded plastic pressure vessel does not exhibit yellowing as compared to a molded article made by an identical process wherein the molten mixture is free of any antioxidant.
